(19) United States
(12) Patent Application Publication
(10) Pub. No.: US 2021/0369943 A1
(43) Pub. Date: Dec. 2, 2021

(54) VESSEL AND DEVICE FOR RECOVERING AND PREPARING ADIPOSE TISSUES

(71) Applicant: STEMCIS, SAINT DENIS (FR)

(72) Inventors: Régis ROCHE, POUILLEY LES VIGNES (FR); Julien GAUTHIER, BESANCON (FR)

(21) Appl. No.: 17/286,129

(22) PCT Filed: Oct. 16, 2019
(86) PCT No.: PCT/FR2019/052455

§ 371 Date: Apr. 16, 2021

(30) Foreign Application Priority Data
Oct. 17, 2018 (FR) 1859598

(51) Int. Cl. A61M 1/00

(52) U.S. Cl. CPC A61M 1/892; A61M 1/63; A61M 1/895; A61M 1/80; A61M 1/784; A61M 1/74

(57) ABSTRACT

Disclosed is a vessel for recovering adipose tissues including a body sealed by a lid connectable to a first pipe with a suction cannula, and a second pipe with a suction pump. A removable adapter connects a lid including two orifices, each closed by a stopper that can be pierced or is pre-pierced and self-sealing, and a vent opening with a filter and removable plug. The connection adapter includes two rigid piercing tubes projecting from under the adapter, each piercing tube in continuous fluid relationship with a corresponding nipple projecting from the adapter, each nipple connectable to a corresponding pipe. The two sealed orifices of the lid and the adapter and piercing tubes are arranged so, when the connection adapter is applied to the lid, each piercing tube pierces a corresponding stopper and brings each nipple into correspondence, in a sealed manner, with the internal volume of the vessel.

### CROSS-REFERENCE TO RELATED APPLICATIONS

This application is the U.S. national phase of International Application No. PCT/FR2019/052455 filed Oct. 16, 2019 which designated the U.S. and claims priority to FR 1859598 filed Oct. 17, 2018, the entire contents of each of which are hereby incorporated by reference.

### BACKGROUND OF THE INVENTION

### Field of the Invention

The present invention generally relates to the field of medical and/or aesthetic devices. It more particularly relates to an adipose tissue recovery jar for recovering adipose tissues during a liposuction operation, as well as a device for the recovery of an adipose tissue and the preparation thereof, in particular with a view to reinjection. A method for the adipose tissue recovery and preparation is also presented.

### Description of the Related Art

At the present time, the autologous lipofilling of large volumes higher than 500 cc, in particular for breast and buttocks, is mainly made with devices that do not allow a simple and rapid centrifugation of the adipose tissues collected.

Either the adipose tissues are recovered into a jar, coupled to an aspirator, and these adipose tissues are then collected from the jar to be directly reinjected, without preparation, to the patient, or otherwise, with a preparation, they can be collected from the jar with syringes, which are then centrifuged, and the content of the centrifuged syringes are then reinjected to the patient.

Or the adipose tissues are manually collected, directly with syringes, which are then centrifuged, and the so-treated adipose tissues are then reinjected to the patient.

In either case, several steps of adipose tissue handling are required, these steps being not necessarily adapted to the management of large volumes of adipose tissues in the operating room, in particular volumes higher than 500 cc, going up to 1 or 2 litres, or even more.

Besides, if the adipose tissues are not centrifuged, the efficiency of the “lipofilling” is much lower, in particular due to the fact that oil is reinjected, with any type of cells, living or not, and cell debris, as well as interstitial liquid that remains higher than 25% of the total volume, even after settling. Finally, a great part of the techniques used do not allow remaining in “closed circuit” during the steps of collection, preparation and reinjection of the adipose tissues, hence increased risks as regards asepsis.

### SUMMARY OF THE INVENTION

It is hence desirable to have means making it possible to maintain in “closed circuit” the steps in question and in particular for the centrifugation, while limiting the necessary handlings and increasing the rapidity of execution of the “lipofilling” in the operating room, for large volumes of adipose tissues, and that is what the present invention proposes.

The invention first proposes an adipose tissue recovery jar for the recovery of the adipose tissues during a liposuction operation, said jar comprising a body closed by a lid defining an internal volume that can be coupled, on the one hand, to a first pipe connected to a suction cannula forming a first fluid circuit and, on the other hand, a second pipe connected to a sucking pump forming a second fluid circuit, the internal volume of the jar and the fluid circuits adjacent to the jar being sealed with respect to the external environment.

According to the invention, the jar further comprises an adapter for connecting the pipes to the jar, said adapter being removable from the lid, and the lid of the jar comprises:
two orifices each closed by a pierceable or pre-pierced and self-sealing plug,
a venting opening comprising internally a filter, the venting opening comprising externally a removable obturator for closing or opening said venting opening, the filter separating the internal volume of the jar from the outside when the obturator of the venting opening is in the open position, and

the connection adapter has two main faces, a lower face and an upper face, the lower face being removably applied against the external upper face of the lid, two rigid piercing tubes protruding from under the lower face of the connection adapter, each of the piercing tubes being in continuous fluid relationship with a corresponding nipple protruding from the upper face of the connection adapter, each nipple being intended to be connected to a corresponding pipe, and

the two closed orifices of the lid as well as the connection adapter and the piercing tubes thereof are arranged in such a way that, when the connection adapter is applied against the lid, each of the piercing tubes pierces one of the matching plugs and establishes a sealed communication between each of the nipples and the internal volume of the jar.

The establishment of a sealed communication also means that the internal lumens of the pipes are fluidically connected to the internal volume of the jar and that the two orifices closed by the pierceable or pre-pierced and self-sealing plugs continue to provide the seal of the jar with respect to the external environment. When the connection adapter is not installed on the lid, whether it has never been installed or after its uninstallation, the pierceable or pre-pierced and self-sealing plugs provide the seal of the jar with respect to the external environment.

Other non-limitative and advantageous features of the jar according to the invention, taken individually or according to all the technically possible combinations, are the following:
the connection adapter is a plate,
the suction cannula can be introduced into adipose tissues,
the rigid piercing tubes are made of plastic material,
the rigid piercing tubes are made of metal,
the rigid piercing tubes are hollow needles,
the jar comprises removable locking means between the connection adapter and the lid,
the connection adapter and the lid comprise foolproofing means preventing an inverted connection of the fluid circuits,
the connection adapter and the lid comprise foolproofing means preventing an inverted connection of the fluid circuits, said means being different diameters of the two piercing tubes,
the connection adapter and the pipes comprise foolproofing means preventing an inverted connection of the fluid circuits, said means being different diameters of the two pipes and the two nipples,
a first one of the two nipples is intended to be connected to the first pipe connected to the suction cannula forming the first fluid circuit and the second one of the two nipples is intended to be connected to the second pipe connected to the suction pump forming the second fluid circuit, and an overfill filter is arranged in the internal volume of the jar, under the lid, in the second fluid circuit, and the overfill filter, the lid and the connection adapter are configured in such a way that the piercing tube corresponding to the second fluid circuit does not interfere with said overfill filter,
the overfill filter is a valve controlled in closing by the rising of the level of adipose tissue recovered in the jar,
the overfill filter is a porous filter preventing the passage of the solid and liquid bodies,
the orifice closed by a pierceable or pre-pierced and self-sealing plug of the first fluid circuit is further intended to serve as a jar content extraction orifice when the connection adapter is separated from the lid, an extraction needle being passed through said orifice,
the connection between the lid and the jar body is sealed,
the lid is removable from the jar body,
the lid is screwed on the jar body,
the lid is crimped on the jar body,
the lid is welded to the jar body,
the lid is bond to the jar body,
the jar further comprises a port for the extraction of the jar content, said extraction port being closed by a removable closing device,
the removable closing device of the extraction port comprises at least one of the following elements: a screwable and unscrewable plug, a pierceable or pre-pierced and self-sealing plug, a retractable shut-off valve,
the pierceable or pre-pierced and self-sealing plugs can be pierced by piercing tubes or hollow needles, the removal of the piercing tubes or the hollow needles leading to a closure of the plugs,
the pierceable or pre-pierced and self-sealing plugs are made of an elastomeric material,
the pierceable or pre-pierced and self-sealing plugs are made of silicone,
the pierceable or pre-pierced and self-sealing plugs are pre-pierced,
the connection adapter further comprises a safety valve in the second fluid circuit, said safety valve opening to the external environment when the vacuum in the second fluid circuit becomes higher in absolute value than a determined vacuum threshold,
the determined vacuum threshold is of 0.4 bar (0.4 10⁵ Pa),
the jar is arranged so as to allow a preparation in the jar of the recovered adipose tissues, with a view to their reinjection,
the jar is configured to further allow a preparation of the recovered adipose tissue at least by centrifugation, when the connection adapter has been removed from the jar,
the jar body is made of plastic material,
the jar body is transparent or translucent,
the jar body comprises volume scale graduations for the content of said jar,
the jar lid is made of plastic material,
the connection adapter is made of plastic material,
the jar and the connection adapter thereof have no metal part,
the jar configured to be centrifuged has a jar body with an axial revolution symmetry, in particular cylindrical or conical,
the jar configured to be centrifuged has a circular lid of determined centre, said lid being preferably symmetrical with respect to a plane perpendicular to the lid and passing through said determined centre, in order to reduce the unbalance of the jar during the centrifugation of said jar.

The invention also relates to a device for the recovery of an adipose tissue of a subject, the preparation of the recovered adipose tissue and for allowing the reinjection to the subject of the adipose tissue preparation, said device comprising:
at least one jar as disclosed,
at least one first pipe intended to be connected to a suction cannula and intended to form a first fluid circuit,
at least one suction cannula,
at least one second pipe intended to be connected to a suction pump and intended to form a second fluid circuit,
at least one syringe of 10 to 20 cc intended to allow a reinjection of the adipose tissue preparation.

This device is preferably in the form of a set of articles or a kit according to all the technical configurations described.

### BRIEF DESCRIPTION OF THE DRAWINGS

The following description in relation with the appended drawings, given by way of non-limitative examples, will allow a good understanding of what the invention consists of and of how it can be implemented.

In the appended drawings:

FIG. 1 is a schematic view of a system for the recovery of adipose tissues with a recovery jar allowing the preparation of the recovered tissue,

FIG. 2 is a schematic view of the functionalities of a jar allowing the preparation of the recovered tissue,

FIG. 3 is a perspective view of a particular example of jar in its functional relationship with different means of the system,

FIGS. 4 and 5 are top and B-B cross-sectional views, respectively, of the jar of FIG. 3, and

FIGS. 6 and 7 are top and C-C cross-sectional views, respectively, of the jar of FIG. 3.

### DESCRIPTION OF THE PREFERRED EMBODIMENTS

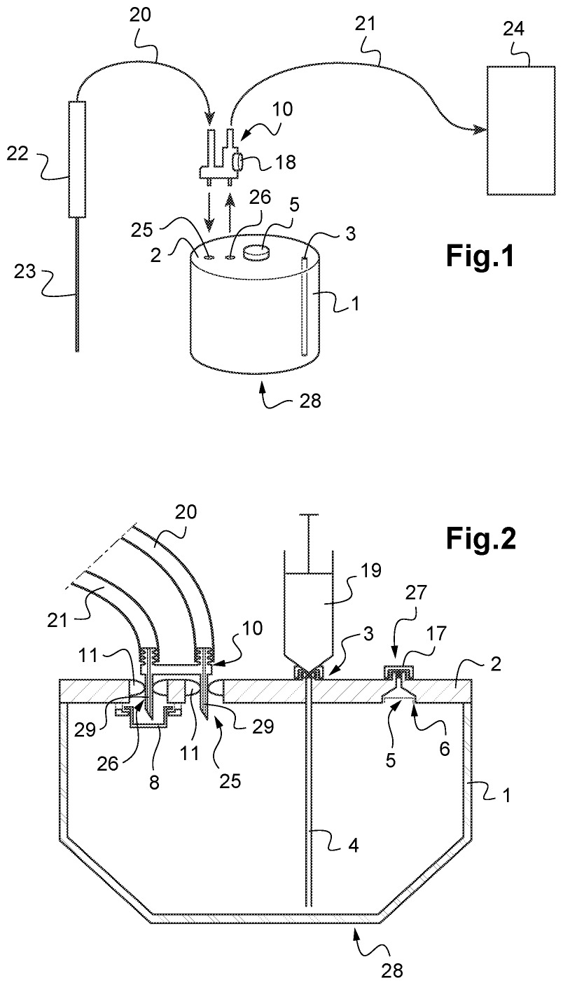

In FIG. 1 is schematically shown an adipose tissue recovery system that comprises a tissue recovery jar 28, schematically shown in FIG. 2, also serving for the preparation of the recovered tissue before the later use of the prepared tissue, in particular for a reinjection. The adipose tissue recovery into the jar generally occurs during a liposuction. This recovery system comprises a cannula 23 on a handle 22 and that is connected to a first pipe 20. This latter 20 is also connected to a connection adapter 10 able to be plugged into a lid 2 closing a body 1 of the jar 28. The lid 2 comprises normally-closed passages allowing access to the internal volume of the jar 28 and, in particular, a first orifice 25 closed by a pierceable and self-sealing plug 11, a second orifice 26 closed by a pierceable and self-sealing plug, a venting opening 27 comprising internally a filter 5 and externally a removable obturator 17, and a jar content extraction port 3 closed by a removable closing device. The connection adapter 10 is also connected to a second pipe 12 connected to a suction pump 24.

The connection adapter 10 allows the passage of two separated fluid circuits each corresponding to one of the two pipes. The first fluid circuit of the first pipe 20 allows the passage of the tissues sucked-up by the cannula 23. The second fluid circuit of the second pipe 21 allows the sucking up and the creation of a vacuum in the jar 28 and the cannula when the connection adapter 10 is plugged into the jar lid 2. Indeed, the connection adapter 10 comprises two tubes 29 for piercing the two plugs 11, each piercing tube corresponding to one of the fluid circuits, and the connection adapter 10 and the lid 2 are configured so that, with the adapter 10 plugged in, the piercing tube of the first fluid circuit pierces the plug 11 of the first orifice 25 and the piercing tube of the second fluid circuit pierces the plug 11 of the second orifice 26. The plugs 11 may be partially or totally pre-pierced in order to allow the passage of the piercing tubes 29. In any case, when the piercing tube 29 or any other piercing object such as a needle is removed from the plug 11, the latter closes up due to the fact that it is self-sealing.

In order to avoid that recovered tissue can pass to the pump 24, an overfill filter is placed in the jar 28 and in relation with the second fluid circuit, i.e., here, under the second orifice 26. This overfill filter is here a valve 8 controlled in closing by the rising of the level of recovered tissue in the jar. The valve 8 forms a shut-off valve.

In order to avoid a too high suction vacuum in the cannula 23, the connection adapter 10 comprises a safety valve 18 in the second fluid circuit. The safety valve 18 opens to the external environment when the vacuum in the second fluid circuit becomes, in absolute value, higher than 0.4 bar (0.4 10⁵ Pa). This safety valve 18 also avoids that the pump vainly sucks up in a second fluid circuit that is closed by the closure of the valve 8 due to filling of the jar and creates therein an excessive vacuum. Indeed, this valve 8 closure will cause the opening of the safety valve 18 because the vacuum in the second fluid circuit will increase. The safety valve 18 is located after the jar, in the second fluid circuit, in such a way that the surrounding air does not enter the jar 28. An audible device, in particular a safety valve suction whistle, or a visual device can be provided to inform the operator that the safety valve 18 has been open and that a jar fill have probably been reached.

The extraction port 3 allows the installation of a syringe 19 for extracting content from the jar, in particular recovered tissue and, after the latter has been treated at least by centrifugation in the jar, for its later use in reinjection. In one embodiment, an extraction tube 4 is installed in the jar 28, connected to the extraction port 3, and a simple syringe 19 is sufficient to extract treated tissue. In another embodiment without such an extraction tube, a syringe is used with comprises a hollow needle that is introduced into the extraction port 3. The extraction port 3 is normally closed by a removable closing device, for example and according to the case, a screwable/unscrewable plug or a pierceable and self-sealing plug or a shut-off valve that retracts upon introduction of the syringe and/or the potential needle thereof.

In an alternative embodiment, the extraction port 3 can be omitted and the extraction of the jar content is then made using a syringe with a needle passed through the plug 11 of the first orifice 25 and after the connection adapter 13 has been removed from the jar lid 2.

Given that the jar 28 is normally sealed when the connection adapter 10 has been removed from the jar lid 2, the venting opening 27 is hence provided, which is open during the extraction of the jar content by the syringe 19 in such a way that air filtered by the filter 5 replaces the extracted tissue. The filter 5 is installed on a fastening ring 6. The filter is a filter 0.22 μm in order to avoid a contamination of the tissues of the jar with potentially non-pure air from the environment.

In order to facilitate the implementation of the system, it is proposed a set of articles, or a kit, intended for a recovery of an adipose tissue and a preparation of the recovered adipose tissue, then for allowing the reinjection of the adipose tissue preparation. This kit intended to treat a subject is sterilized and single-use, and it typically comprises:
from one to four jars 28 intended to receive the adipose tissue then to allow the preparation thereof, in particular by centrifugation in a centrifuge adapted to the jar. The jars comprise normally closed openings.
a connection adapter 10.
a cannula 23 connected to a handle.
at least the pipe 20 of the first fluid circuit and, preferably, the two pipes 20, 21.
an extraction tube 4 in the case where the latter would not be integrated to the lid 2 of the jar 28.
syringes from 10 to 20 cc for the reinjection of the treated tissue. Injection or infiltration needles or cannulas, according to the case, intended to be installed on the syringes for the reinjection of the treated tissue, may further be provided.

The jar, filled with recovered adipose tissue, without the connection adapter, can be centrifuged at 900 g, to treat the recovered tissue. A previous washing of the recovered tissues is possible before the centrifugation. It is potentially possible to introduce one or several preparation products in the jar.

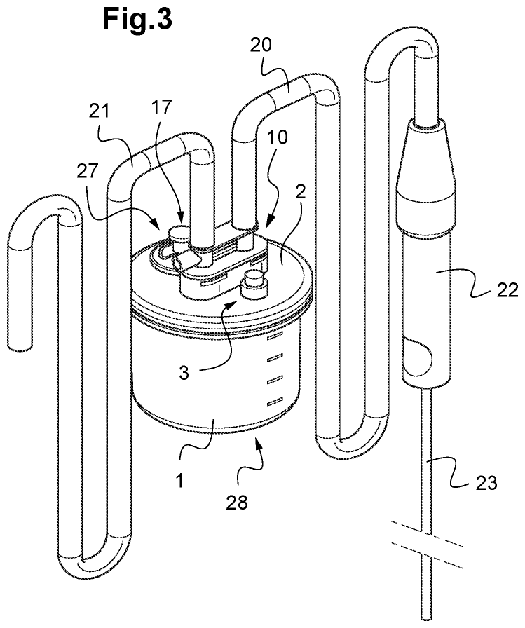

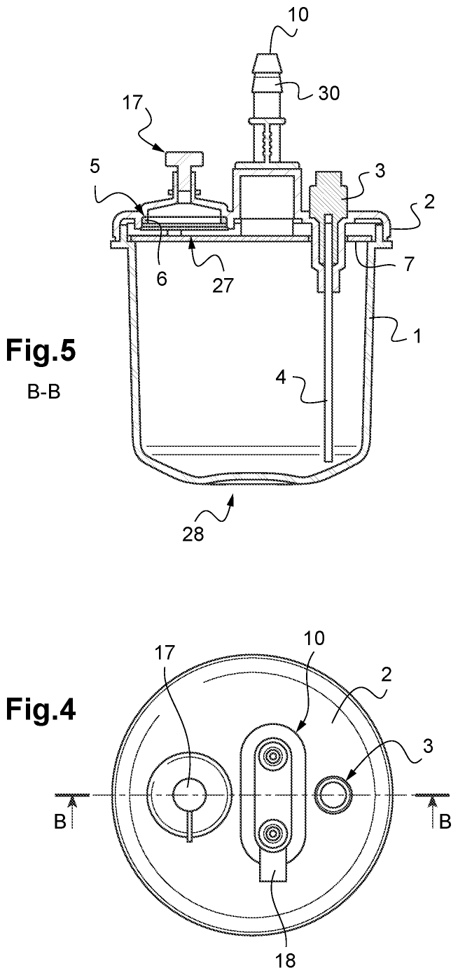

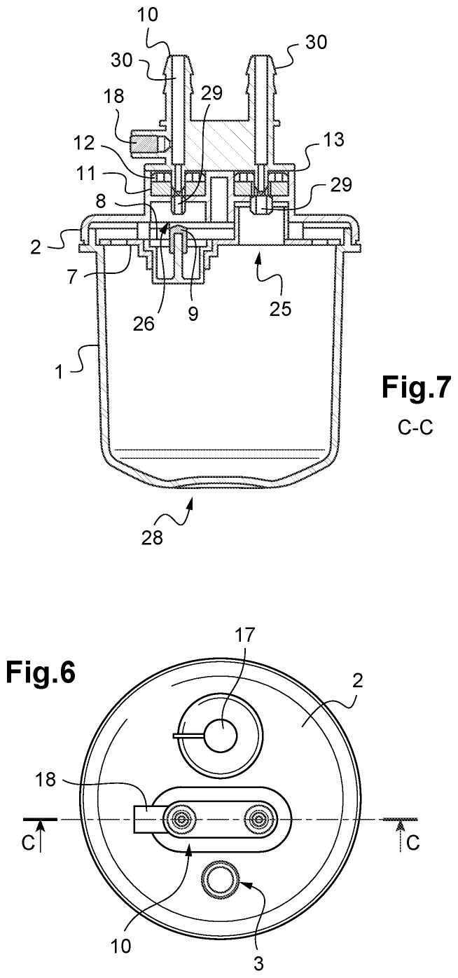

A concrete example of jar adapted to the system of the invention will now be described with FIGS. 3 and following. The shown jar 28 results from the assembly of several parts made of plastic material, comprising in particular a body 1 and a lid 2. The jar 28 hence has a body 1 of axial revolution symmetry that is almost cylindrical, which is closed at the bottom and open at the top. The top of the body 1 is closed by the lid 2, the connection between the body 1 and the lid 2 being preferably welded to provide a perfect seal between both. A support plate 7 is fastened under the lid, in the jar 28, in order to maintain certain of the elements of the lid, comprising in particular the filter 5, the overfill valve 8. The lid 2 comprises the first orifice 15 closed by a pierceable or pre-pierced and self-sealing plug 11, the second orifice 26 closed by a pierceable or pre-pierced and self-sealing plug 11 with, underneath, the overfill valve 8, the venting opening 27 with, internally, its filter 5+fastening ring 6 and, externally, the removable obturator 17, and the extraction port 3 closed by a removable closing device. In this example, an extraction tube 4 is integrated to the lid. Preferably, the extraction port 3 comprises a standard screwing system for the fastening of the syringe end tip, in particular of the “Luer-loc®” type.

In FIGS. 3 and following, the connection adapter 10 is plugged into the lid 2 of the jar 28 and its two piercing tubes 29, oriented downward, pass through the plugs 11 of the two orifices 25, 26. The connection adapter 10 is wholly in the form of a plate that is fastened to the jar lid.

The two piercing tubes 29 have different shapes and/or sizes adapted to each of the two orifices 25, 26 in order to create a foolproofing. More precisely, two pierced wafers of different internal diameters are arranged in the two orifices 25, 26 and they further serve to hold the plugs 11 in place. In fluid relationship with the piercing tubes 29, the connection adapter 10 comprises, towards the top, two nipples 30 for the connection to the two pipes 20, 21. FIG. 7 is a cross-sectional view of the safety valve 18 of the second fluid circuit, that which is connected to the suction pump 24.

The use of the jar according to the invention can be made according to the following example. The operator connects the cannula 23 and the handle 22 thereof to the nipple 30 of the first fluid circuit of the connection adapter 10 thanks to the first pipe 20. The operator connects the pump 24 to the nipple 30 of the second fluid circuit of the connection adapter 10 thanks to the second pipe 21. The operator inserts/plugs the connection adapter 10 into the lid 2 of the jar 28 while respecting the orientation imposed by the foolproofing means if the jar used had not already the connection adapter 10 inserted/plugged into it. It can be noted that it is preferable that the jar is provided with the adapter 10 separated from the jar 28 due to the fact that, in the absence of adapter, the jar is normally closed. The order of these operations can be modified according to the needs.

The operator operates the pump and proceeds to the suction of the adipose tissues that are then deposited into the jar. Once the jar full, which can be visually detected with a transparent or translucent jar, or due to the closure of the overfill valve 8 causing the opening of the safety valve 18, the operator, preferably after having stopped the pump, can change the jar by passing the connection adapter 10 to an empty jar, then continue the suction of the adipose tissues. The number of jar/jars to be filled depends on the need for reinjection.

The following steps are performed on the filled jars and without the connection adapter.

The preparation of the adipose tissues recovered in a jar can begin by a washing and it comprises a centrifugation of the jar. During this preparation, the jar closes up due to the fact that the plugs 11 are of the self-sealing type once the connection adapter 10 removed from the lid 2: they automatically close up. The prepared tissues useful for the reinjection end up at the bottom of the jar after centrifugation, hence the length of the extraction tube 4 in FIGS. 2 and 5.

Then, the operator opens the removable closing device from the extraction port 3 and fastens a syringe 19 thereto, then he opens the removable obturator 17 of the venting opening 27 to allow the equalization of the pressures between the internal volume of the jar and the outside during the suction of the prepared tissues by the syringe. It is to be noted that it is highly preferably to firstly open the venting opening 27, which comprises a filter 5, before opening the extraction port 3 to avoid that air enters the jar through the extraction port 3 in case of previous internal vacuum of the jar. It can however be provided on the extraction port a valve system controlled by the installation of the syringe, to limit this risk.

Once the syringe filled with the prepared tissue, it can be removed from the lid, preferably the extraction port is then closed up, and an injection or infiltration needle or cannula is fastened to the syringe so that the reinjection can be performed.

1. An adipose tissue recovery jar (28) for the recovery of the adipose tissues during a liposuction operation, said jar (28) comprising a body (1) closed by a lid (2) defining an internal volume that can be coupled, to a first pipe (20) connected to a suction cannula (23) forming a first fluid circuit and, also to a second pipe (21) connected to a sucking pump (24) forming a second fluid circuit, the internal volume of the jar (28) and the fluid circuits adjacent to the jar (28) being sealed with respect to the external environment,
the jar comprising an adapter (10) for the connection of the pipes (20, 21) to the jar (28), said adapter (10) being removable from the lid (2), wherein the lid (2) of the jar (28) comprises:
two orifices (25, 26) each closed by a pierceable or pre-pierced and self-sealing plug (11),
a venting opening (27) comprising internally a filter (5), the venting opening (27) comprising externally a removable obturator (17) for closing or opening said venting opening (27), the filter (5) separating the internal volume of the jar (28) from the outside when the obturator (17) of the venting opening (27) is in the open position, and
wherein the connection adapter (10) has two main faces, a lower face and an upper face, the lower face being removably applied against the external upper face of the lid (2), two rigid piercing tubes (29) protruding from under the lower face of the connection adapter (10), each of the piercing tubes (29) being in continuous fluid relationship with a corresponding nipple (30) protruding from the upper face of the connection adapter (10), each nipple (30) being intended to be connected to a corresponding pipe (20, 21), and
wherein the two closed orifices (25, 26) of the lid (2) as well as the connection adapter (10) and the piercing tubes (29) thereof are arranged in such a way that, when the connection adapter (10) is applied against the lid (2), each of the piercing tubes (29) pierces one of the matching plugs (11) and establishes a sealed communication between each of the nipples (30) and the internal volume of the jar (28).

2. The jar (28) according to claim 1, further comprising a removable locking means between the connection adapter (10) and the lid (2).

3. The jar (28) according to claim 1, wherein a first one of the two nipples (30) is intended to be connected to the first pipe (20) connected to the suction cannula (23) forming the first fluid circuit and the second one of the two nipples (30) is intended to be connected to the second pipe (21) connected to the suction pump (24) forming the second fluid circuit, and wherein an overfill filter is arranged in the internal volume of the jar (28), under the lid (2), in the second fluid circuit, and wherein the overfill filter, the lid (2) and the connection adapter (10) are configured in such a way that the piercing tube (29) corresponding to the second fluid circuit does not interfere with said overfill filter.

4. The jar (28) according to claim 3, wherein the overfill filter is a valve (8) controlled in closing by the rising of the level of adipose tissue recovered in the jar (28).

5. The jar (28) according to claim 1, further comprising (3) for the extraction of the jar (28) content, said extraction port (3) being closed by a removable closing device.

6. The jar (28) according to claim 5, characterized in that the removable closing device of the extraction port (3) comprises at least one of the following elements: a screwable and unscrewable plug, a pierceable or pre-pierced and self-sealing plug, a retractable shut-off valve.

7. The jar (28) according to claim 1, wherein the connection adapter (10) further comprises a safety valve (18) in the second fluid circuit, said safety valve (18) opening to the external environment when the vacuum in the second fluid circuit becomes higher in absolute value than a determined vacuum threshold.

8. The jar (28) according to claim 1, wherein the determined vacuum threshold is of 0.4 bar (0.4 10⁵ Pa).

9. The jar (28) according to claim 1, wherein the jar is configured to further allow a preparation of the recovered adipose tissue at least by centrifugation, when the connection adapter (10) has been removed from the jar (28).

10. A device for the recovery of an adipose tissue of a subject, the preparation of the recovered adipose tissue and for allowing the reinjection to the subject of the adipose tissue preparation, said device comprising:
at least one jar (28) according to claim 1,
at least one first pipe (20) intended to be connected to a suction cannula (23) and intended to form a first fluid circuit,
at least one suction cannula (23),
at least one second pipe (21) intended to be connected to a suction pump (24) and intended to form a second fluid circuit,
at least one syringe (19) of 10 to 20 cc intended to allow a reinjection of the adipose tissue preparation.

11. The jar (28) according to claim 2, wherein a first one of the two nipples (30) is intended to be connected to the first pipe (20) connected to the suction cannula (23) forming the first fluid circuit and the second one of the two nipples (30) is intended to be connected to the second pipe (21) connected to the suction pump (24) forming the second fluid circuit, and wherein an overfill filter is arranged in the internal volume of the jar (28), under the lid (2), in the second fluid circuit, and wherein the overfill filter, the lid (2) and the connection adapter (10) are configured in such a way that the piercing tube (29) corresponding to the second fluid circuit does not interfere with said overfill filter.

12. The jar (28) according to claim 2, further comprising (3) for the extraction of the jar (28) content, said extraction port (3) being closed by a removable closing device.

13. The jar (28) according to claim 3, further comprising (3) for the extraction of the jar (28) content, said extraction port (3) being closed by a removable closing device.

14. The jar (28) according to claim 4, further comprising (3) for the extraction of the jar (28) content, said extraction port (3) being closed by a removable closing device.

15. The jar (28) according to claim 2, wherein the connection adapter (10) further comprises a safety valve (18) in the second fluid circuit, said safety valve (18) opening to the external environment when the vacuum in the second fluid circuit becomes higher in absolute value than a determined vacuum threshold.

16. The jar (28) according to claim 3, wherein the connection adapter (10) further comprises a safety valve (18) in the second fluid circuit, said safety valve (18) opening to the external environment when the vacuum in the second fluid circuit becomes higher in absolute value than a determined vacuum threshold.

17. The jar (28) according to claim 4, wherein the connection adapter (10) further comprises a safety valve (18) in the second fluid circuit, said safety valve (18) opening to the external environment when the vacuum in the second fluid circuit becomes higher in absolute value than a determined vacuum threshold.

18. The jar (28) according to claim 5, wherein the connection adapter (10) further comprises a safety valve (18) in the second fluid circuit, said safety valve (18) opening to the external environment when the vacuum in the second fluid circuit becomes higher in absolute value than a determined vacuum threshold.

19. The jar (28) according to claim 6, wherein the connection adapter (10) further comprises a safety valve (18) in the second fluid circuit, said safety valve (18) opening to the external environment when the vacuum in the second fluid circuit becomes higher in absolute value than a determined vacuum threshold.

20. The jar (28) according to claim 2, wherein the determined vacuum threshold is of 0.4 bar (0.4 105 Pa).
